(19) United States
(12) Patent Application Publication
(10) Pub. No.: US 2010/0179659 A1
(43) Pub. Date: Jul. 15, 2010

(54) CELL-NANOFIBER COMPOSITE AND CELL-NANOFIBER-HYDROGEL COMPOSITE AMALGAM BASED ENGINEERED INTERVERTEBRAL DISC

(76) Inventors: Wan-Ju Li, Madison, WI (US); Leon J. Nesti, Crownsville, MD (US); Rocky S. Tuan, Pittsburgh, PA (US)

Correspondence Address: EDWARDS ANGELL PALMER & DODGE LLP, PO BOX 55874, BOSTON, MA, 02205, US

(21) Appl. No.: 12/443,393

(22) PCT Filed: Sep. 27, 2007
(86) PCT No.: PCT/US07/20974

§ 371 Date: Feb. 26, 2010

Related U.S. Application Data
(60) Provisional application No. 60/847,839, filed on Sep. 27, 2006.
(60) Provisional application No. 60/848,284, filed on Sep. 28, 2006.

(51) Int. Cl. A61F 2/44; C12N 5/00

(52) U.S. Cl. 623/17.16; 435/397

(57) ABSTRACT

The instant invention is directed to a tissue engineered intervertebral disc comprising at least one inner layer and an exterior layer, wherein: the exterior layer comprises a nanofibrous polymer support comprising one or more polymer nanofibers; the at least one inner layer comprises a hydrogel composition comprising at least one or more hydrogel materials and/or one or more polymer nanofibers; and a plurality of cells which are dispersed throughout the tissue engineered intervertebral disc. Additionally, the instant invention is directed to methods of making such intervertebral discs and methods of treating intervertebral disc damage.

### CROSS-REFERENCE TO RELATED APPLICATION

This application claims the benefit of U.S. provisional application No. 60/847,839 filed Sep. 27, 2006 and U.S. provisional application No. 60/848,284 filed Sep. 28, 2006, both of which are fully incorporated herein by reference.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services.

### FIELD OF INVENTION

The present invention relates to tissue engineered intervertebral discs comprising a nanofibrous polymer hydrogel amalgam having cells dispersed therein, methods of fabricating tissue engineered intervertebral discs by culturing a mixture of stem cells or intervertebral disc cells and a electrospun nanofibrous polymer hydrogel amalgam in a suitable bioreactor, and methods of treatment comprising implantation of tissue engineered intervertebral disc into a subject.

### BACKGROUND OF THE INVENTION

Diseased or damaged tissue has often been replaced by an artificial material, cadaver tissue, or donated, allogenic tissue. Tissue engineering offers an attractive alternative whereby a live, natural tissue/support composition is generated from a construct made up of a subject's own cells in combination with a scaffold for replacement of defective tissue.

Degeneration of the intervertebral disc (IVD) is a common and significant source of morbidity in our society. Approximately 8 of 10 adults at some point in their life will experience an episode of significant low back pain, with the majority improving without any formal treatment. However, for the subject requiring surgical management current interventions focus on fusion of the involved IVD levels, which eliminates pain but does not attempt to restore disc function (Shvartzman, L. et al. (1992) Spine 17(2), 176-182). Approximately 200,000 spinal fusions were performed in the United States in 2002 to treat pain associated with lumbar disc degeneration. Spinal fusion however is thought to significantly alter the biomechanics of the disc and lead to further degeneration, or adjacent segment disease. Therefore, in the past decade there has been mounting interest in the concept of IVD replacement (Deyo, R. A. and Tsui-Wu, Y. J. (1987) Spine 12(3), 264-268). The replacement of the IVD holds tremendous potential as an alternative to spinal fusion for the treatment of degenerative disc disease by offering a safer alternative to current spinal fusion practices.

At the present time, several disc replacement implants are at different stages of preclinical and clinical testing. These disc replacement technologies are designed to address flexion, extension, and lateral bending motions; however, they do little to address compressive forces and their longevity is limited due to their inability to biointegrate. Therefore, a cell-based tissue engineering approach offers the most promising alternative to replace the degenerated IVD. Current treatment for injuries that penetrate subchondral bone include subchondral drilling, periosteal tissue grafting, osteochondral allografting, chondrogenic cell and transplantation; but are limited due to suboptimal integration with host tissues.

Cell-based tissue engineering is a burgeoning field that utilizes cells on or within a synthetic scaffolding material toward the fabrication of functional biological substitutes for the replacement of lost or damaged tissues (Langer, R. and Vacanti, J. P. (1993) Science 260 (5110), 920-926). For cell-based tissue engineering to succeed cells need to interact with an appropriate scaffolding material, which is able to closely mimic the structure, biologic, and mechanical function of the native extracellular matrix (ECM) found in tissues. This artificial ECM provides a three-dimensional substrate for cells to form new tissues with appropriate structure and function, and can also enable the delivery of cells and appropriate bioactive factors. Eventually, these artificial matrices will degrade and be replaced by the ECM proteins secreted by the ingrowing cells. The ultimate goal of cell-based tissue engineering is to fabricate biologically compatible tissues that over time will fully integrate into the human body.

In order to achieve this goal, a scaffolding material must be properly designed to ensure biocompatibility with the seeded cells. Nanofibrous scaffolds (NFS) have recently received a great deal of attention as novel scaffolds that closely mimic the architectural scale and morphology of collagen fibrils comprising the natural ECM. To date, three various techniques have been utilized to fabricate NFS, which are: electrospinning (Li, W. J. et al. (2002) J Biomed Mater Res 60(4), 613-621), phase separation (Ma, P. X., and Zhang, R. (1999) J Biomed Mater Res 46(1), 60-72), and self-assembly (Zhang, S. et al. (2002) Curr Opin Chem Biol 6(6), 865-871). The electrospinning method has been used to fabricate non-woven, three-dimensional, porous, nano-scale fiber-based scaffolds for various tissue engineering applications (Venugopal, J., and Ramakrishna, S. (2005) Tissue Eng 11(5-6), 847-854; Riboldi, S. A. et al. (2005) Biomaterials 26(22), 4606-4615; Lee, C. H. et al. (2005) Biomaterials 26(11), 1261-1270; Li, W. J. et al. (2003) J Biomed Mater Res A 67(4), 1105-1114). The characteristic features of NFS are that they morphologically mimic the native ECM with its abundant collagen fibrils, have a high porosity (90%), have favorable mechanical properties, high surface area-to-volume ratio, and a wide range of pore size distribution (Li, W. J. et al. (2002) J Biomed Mater Res 60(4), 613-621).

In order to further enhance the likeness of the electrospun NFS with the native ECM an amalgam was developed using NFS and hyaluronic acid (HA). HA is a glycosaminoglycan that plays an integral role as a lubrication proteoglycan in the native ECM. HA is able to provide structural support and provide biochemical cues during cellular differentiation and proliferation (Lisignoli, G. et al. (2006) J Biomed Mater Res A 77(3), 497-506). For example, it has been shown that HA stimulates chondrogenesis of embryonic mesenchymal progenitor cells (Hwang, N. S. et al. (2006) Biomaterials 27(36), 6015-6023).

The IVD is comprised of two distinct anatomic regions, the annulus fibrosus (AF) and the nucleus pulposus (NP), which are sandwiched between two cartilaginous endplates and bony vertebral bodies. In IVD tissue engineering, the NP and AF cells have been extensively studied in their potential to regenerate the two distinct regions of the IVD (Kluba, T. et al. (2005) Spine 30(24), 2743-2748). However, few studies have investigated the potential of mesenchymal stem cells (MSCs) in IVD tissue engineering. Under the proper conditions, MSCs may provide a more ideal cell source for the regeneration of the two distinct regions of the IVD. MSCs are multipotential cells capable of giving rise to cells of mesenchymal origin including osteoblasts, myoblasts, annulus fibrosus cells, nucleus pulposus cells, adipocytes, and tendon cells. MSCs provide an ideal cell source for IVD tissue engineering for the following reasons: (1) they are generally considered to be easily accessible and readily available, (2) they possess extensive self-renewal or expansion capability, and (3) they possess little to no immunogenic or tumorgenic ability. All of these criteria are well suited for an ideal cell source for cell-based tissue engineering.

Through the application of the ideal cell type within the appropriate scaffolding material, surgeons can overcome current limitations in the surgical treatment of degenerative disc disease in order to profoundly improve clinical outcomes.

### SUMMARY OF THE INVENTION

In one aspect, the invention provides a tissue engineered intervertebral disc, comprising: a nanofibrous polymer support comprising one or more polymer nanofibers; a hydrogel composition comprising at least one or more hydrogel materials; and a plurality of cells which are dispersed throughout the tissue engineered intervertebral disc.

In another aspect, the invention provides a tissue engineered intervertebral disc comprising at least one inner layer and an exterior layer, wherein: the exterior layer comprises a nanofibrous polymer support comprising one or more polymer nanofibers; the at least one inner layer comprises a hydrogel composition comprising at least one or more hydrogel materials; and a plurality of cells which are dispersed throughout the tissue engineered intervertebral disc.

In another aspect, the invention provides a tissue engineered intervertebral disc comprising at least one inner layer and an exterior layer, wherein: the exterior layer comprises a nanofibrous polymer support comprising one or more polymer nanofibers; the at least one inner layer comprises a hydrogel composition comprising at least one or more hydrogel materials and one or more polymer nanofibers; and a plurality of cells which are dispersed throughout the tissue engineered intervertebral disc.

In one aspect, the invention provides a method of preparing a tissue engineered intervertebral disc comprising the steps of: preparing a nanofibrous biocompatible polymer support comprising a cavity; contacting a suspension of cells with the surface of the support to form a polymer matrix having cells dispersed therein; injecting a hydrogel composition into the cavity; and culturing the cell-polymer matrix in a bioreactor with a culture medium under conditions conducive to growth of cells into a tissue engineered intervertebral disc.

In another aspect, the invention provides a method of forming intervertebral disc in vivo, the method comprising the steps of: preparing the tissue engineered intervertebral disc of the invention; and inserting the tissue engineered intervertebral disc into a subject at the position suitable for formation of new intervertebral disc.

In another aspect, the invention provides a method of treating intervertebral disc damage, the method comprising the steps of: preparing the tissue engineered intervertebral disc of the invention; and inserting the tissue engineered intervertebral disc into a subject at the location of the damaged intervertebral disc.

In another aspect, the invention provides a method for treating intervertebral disc damage, the method comprising the steps of: harvesting annulus fibrosus cells, nucleus pulposus cells, mesenchymal stem cells, or embryonic stem cells from a subject; preparing tissue engineered intervertebral disc of the invention, wherein the cells are the annulus fibrosus cells, nucleus pulposus cells, mesenchymal stem cells, or embryonic stem cells harvested from the subject; implanting the tissue engineered intervertebral disc in the subject in a locus having damaged intervertebral disc.

In still another aspect, the invention provides a method for cosmetic or reconstructive surgery, the method comprising the steps of: preparing the tissue engineered intervertebral disc of the invention; and inserting the tissue engineered intervertebral disc into a subject.

In yet another aspect, the invention provides a method for cosmetic or reconstructive surgery, the method comprising the steps of: harvesting annulus fibrosus cells, nucleus pulposus cells, mesenchymal stem cells, or embryonic stem cells from a subject; preparing tissue engineered intervertebral disc of the invention, wherein the cells are the annulus fibrosus cells, nucleus pulposus cells, mesenchymal stem cells, or embryonic stem cells harvested from the subject; and implanting the tissue engineered intervertebral disc in the subject in a locus having damaged intervertebral disc.

In another aspect, the invention provides a method of preparing tissue engineered intervertebral disc comprising the steps of: preparing a nanofibrous biocompatible polymer support comprising a cavity; contacting a suspension of cells with the surface of the support to form a polymer matrix having cells dispersed therein; injecting a hydrogel composition into the cavity; and culturing the cell-polymer matrix in a bioreactor with a culture medium under conditions conducive to cell growth and differentiation to tissue engineered tissue.

In yet another aspect, the invention provides a method of preparing a tissue engineered tissue comprising the steps of: preparing a nanofibrous biocompatible polymer support comprising a cavity; expanding the nanofibrous polymer support thereby increasing interfiber distance; contacting a suspension of cells with the support to form a polymer matrix having cells dispersed therein; injecting a hydrogel composition into the cavity; culturing the compressed cell-polymer matrix in a bioreactor with a culture medium under conditions to conducive cell growth and differentiation to tissue engineered tissue.

In certain aspects, a cell-based tissue engineering approach was utilized to develop a novel hyaluronic acid-nanofiber amalgam to engineer two regions of the IVD using human bone marrow-derived mesenchymal stem cells.

### BRIEF DESCRIPTION OF THE DRAWINGS

For a more complete understanding of the nature and desired objects of the present invention, reference is made to the following detailed description taken in conjunction with the accompanying drawing figures wherein like reference character denote corresponding parts throughout the several views and wherein:

FIG. 1 is a schematic of an electrospinning apparatus for the preparation of nanofibrous polymer supports suitable for use in the invention;

FIG. 2. is a drawing of a hollow nanofibrous polymer shaped as a cylinder; A represents the nanofibrous polymer support; B represents a hollow cavity;

FIG. 3. is a drawing of a cross section of a nanofibrous polymer shaped as a cylinder, wherein the cavity is filled with “cotton ball” nanofibers; C represents the “cotton ball” nanofibers;

FIG. 4. is a drawing of a cross section of a nanofibrous polymer shaped as a cylinder, wherein the cavity is filled with “cotton ball” nanofibers, wherein the ends of the nanofibrous polymer support are sealed with a sealant D;

FIG. 5. is a drawing of a cross section of a nanofibrous polymer support comprising a cavity, that is sealed with a sealant D, and injected with a hydrogel composition E into the cavity;

FIG. 6. IVD-NFS after 7 days in culture. Alcian blue staining at both low (1) and high (2) magnification demonstrates proteoglycan deposition in both the outer annulus and inner nucleus portion of the disc. H&E staining demonstrates abundant cell population of the annulus and fewer cells in the nucleus at both low (3) and high (4) magnification;

FIG. 7. IVD NFS after 14 days in culture. Note increasing proteoglycan production throughout the construct at both low (1) and high (2) magnification evident by alcian blue staining. H&E staining demonstrates flattened cell type in the periphery and more rounded cell in the center (3,4);

FIG. 8. IVD NFS after 28 days in culture. Alcian blue staining permeates construct (1,2). Note more even distribution of cell population in both inner and outer regions (3,4). Cells continue to be spindle shaped in periphery and more rounded in the center;

FIG. 9. Immunohistochemistry for col I (the first row), col II (the second row), aggrecan (the third row), and link protein (the fourth row) after 7 (the first column), 14 (the second column), and 28 (the third column) days in culture. There are steady increase in ECM expression in both the annulus fibrosus (AF) and nucleus pulposus (NP);

FIG. 10. Scanning electron microscopy of the AF (the first column) and NP (the second column) over the 28 day period;

FIG. 11. Gel electrophoresis of RNA extracts from region of the AF and NP after 7, 14, and 28 days in culture. Lane 1=col I, Lane 2=col II, Lane 3=col IX, Lane 4=col X, Lane 5=col XI, Lane 6=aggrecan, and Lane 7=COMP;

FIG. 12. GAG analysis of HANFS constructs at 7, 14, and 21 days. There is a significant increase in the sulfated GAG production from day 7 to 14. The GAG production demonstrates further increase from day 14 to 21 (p<0.05); however this increase does not reach statistical significance (p=0.119).

### DETAILED DESCRIPTION

Although a preferred embodiment of the invention has been described using specific terms, such description is for illustrative purposes only, and it is to be understood that changes and variations may be made without departing from the spirit or scope of the following claims.

Methods and materials to form an intervertebral disc, are described wherein cells, e.g., annulus fibrosus cells, nucleus pulposus cells or stem cells, are seeded onto or into a nanofibrous polymer-hydrogel composition, which cell-polymer-hydrogel matrix is then cultured in a rotating bioreactor to form the intervertebral disc. The product intervertebral disc generated in the methods of the invention is implantation into a subject in therapeutic, prophylactic or cosmetic procedures.

### Tissue Engineered Intervertebral Disc

In one aspect, the invention provides a tissue engineered intervertebral disc, comprising: a nanofibrous polymer support comprising one or more polymer nanofibers; a hydrogel composition comprising at least one or more hydrogel materials; and a plurality of cells which are dispersed throughout the tissue engineered intervertebral disc.

In another aspect, the invention provides a tissue engineered intervertebral disc comprising at least one inner layer and an exterior layer, wherein: the exterior layer comprises a nanofibrous polymer support comprising one or more polymer nanofibers; the at least one inner layer comprises a hydrogel composition comprising at least one or more hydrogel materials; and a plurality of cells which are dispersed throughout the tissue engineered intervertebral disc.

In another aspect, the invention provides a tissue engineered intervertebral disc comprising at least one inner layer and an exterior layer, wherein: the exterior layer comprises a nanofibrous polymer support comprising one or more polymer nanofibers; the at least one inner layer comprises a hydrogel composition comprising at least one or more hydrogel materials and one or more polymer nanofibers; and a plurality of cells which are dispersed throughout the tissue engineered intervertebral disc.

In certain embodiments, the invention provides a tissue engineered intervertebral disc, wherein the nanofibrous polymer support is made by electrospinning.

In one embodiment, the nanofibrous polymer support comprises poly(glycolide) (PGA), poly (L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly (ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(3-hydroxybutyrate-co-3-hydroxyvalerate), poly(ethylene-co-vinyl alcohol), and combinations thereof.

In another embodiment, the nanofibrous polymer support comprises biodegradable poly(α-hydroxy ester) polymers. In a further embodiment, the nanofibrous polymer support comprises polymers selected from poly(lactic acid) (PLA), poly(glycolide) (PGA), and poly(lactide-co-glycolide) (PLGA), and combinations thereof.

In other embodiments, the nanofibrous polymer support comprises poly(glycolide) (PGA), poly(lactide-co-glycolide) (PLGA), poly(L-lactide) (PLLA), poly(D,L-lactide) (P(DLLA)), poly(ε-caprolactone) (PCL), and combinations thereof.

In one embodiment, the hydrogel composition comprises a hydrogel selected from non-biodegradable hydrogels, natural biodegradable hydrogels, and synthetic biodegradable hydrogels. In certain embodiments, the hydrogel composition comprises a hydrogel selected from the following: self-assembly peptide, fibrin, alginate, agarose, hyaluronan, hyaluronic acid, chitosan, chondroitin sulfate, polyethylene oxide (PEO), poly(ethylene glycol) (PEG), collagen type I, collagen type II, and combinations thereof. In a further embodiment, the hydrogel composition comprises a hydrogel selected from the following: self-assembly peptide, fibrin, alginate, agarose, hyaluronan, hyaluronic acid, chitosan, chondroitin sulfate, collagen type I, collagen type II, and combinations thereof.

Other suitable hydrogels include bioabsorbable materials selected from gelatin, alginic acid, chitin, chitosan, dextran, polyamino acids, polylysine, and copolymers of these materials. In other aspects, suitable hydrogels include those manufactured from biodegradable materials which degrade in vivo or in vitro, at a sufficiently slow rate to retain the desired nanoscale morphology during the tissue culturing process.

A variety of cells can be used to form engineered tissues. Annulus fibrosus cells, nucleus pulposus cells, mesenchymal stem cells, and embryonic stem cells are generally preferred cells for the preparation of intervertebral discs. Mesenchymal stem cells can be isolated from various tissues, including but not limited to muscle, blood, bone marrow, fat, cord blood, placenta, and other tissues known to contain mesenchymal stem cells. In certain embodiments, nucleus pulposus cells are derived from fibrocartilage, which is expressed from chondrocytes.

In yet another embodiment, the cells are selected from annulus fibrosus cells, nucleus pulposus cells, mesenchymal stem cells, and embryonic stem cells, or combinations thereof. In certain embodiments, each of the annulus fibrosus cells, nucleus pulposus cells, mesenchymal stem cells, and embryonic stem cells dispersed throughout the tissue engineered intervertebral disc is in contact with at least one polymer and at least one other annulus fibrosus cells, nucleus pulposus cell, mesenchymal stem cell, or embryonic stem cell. In other embodiments, each of the annulus fibrosus cells, nucleus pulposus cells, mesenchymal stem cells, and embryonic stem cells dispersed throughout the tissue engineered intervertebral disc is in contact with a plurality of other annulus fibrosus cells, nucleus pulposus mesenchymal stem cells, or embryonic stem cells.

Upon administration of annulus fibrosus cells and nucleus pulposus cells to the nanofibrous polymer support, the cells remain differentiated as the annulus fibrosus cells and nucleus pulposus cells and begin to form the extracellular matrix. Stem cells, including adult mesenchymal stem cells and embryonic stem cells, particularly MSC originating from a subject in need of replacement cartilage are suitable for use in the methods of the invention and differentiate to annulus fibrosus cells and nucleus pulposus cells when the MSC cells are in contact with the nanofibrous polymer-hydrogel compositions used in the methods of the invention. Other collagen generating cells are also contemplated for use in the methods of the invention, including but not limited to tenocytes, ligamentum cells, fibroblasts, and dermal fibroblasts.

In certain aspects where the engineered tissue is intended for implantation into a subject as part of a therapeutic, preventative, or cosmetic surgical procedure, autologous cells obtained by a biopsy are used as seed cells in the methods of engineering tissues or methods of engineering intervertebral discs provided herein. Cells can be obtained directly from a donor, washed and suspended in a culture media before contacting the cells with the nanofibrous polymer-hydrogel. To enhance cell viability, the cells are generally added or mixed with the culture media just prior to incorporation into the nanofibrous polymer support. Cell viability can be assessed using standard techniques including visual observation with a light or scanning electron microscope, histology, or quantitative assessment with radioisotopes. The biological function of the cells incorporated into the nanofibrous polymer-hydrogel scaffold can be determined using a combination of the above techniques.

Cells obtained by biopsy are harvested, cultured, and then passaged as necessary to remove non-cellular contaminants and contaminating, unwanted cells. Annulus fibrosus cells and nucleus pulposus cells are isolated from autologous IVD by excision of tissue, then either enzymatic digestion of cells to yield dissociated cells or mincing of tissue to form explants which are grown in cell culture to yield cells for seeding onto the nanofibrous polymer-hydrogel supports. Mesenchymal stem cells are isolated from autologous bone marrow. Typically bone marrow is harvested from the interior of the femoral neck and head by using a bone curet and then isolated from particulates and other cells (e.g., non-adherent hematopoietic and red blood cells) by centrifugation and exchange of culture medium.

In still another embodiment, the invention provides a tissue engineered intervertebral disc, wherein the hydrogel composition is encapsulated by the polymer support.

In another embodiment, the invention provides a tissue engineered intervertebral disc, wherein the inner layer is encapsulated by the exterior layer. In one embodiment, the inner layer is encapsulated by a sealant. In certain embodiments, the sealant is selected from nanofibrous polymers of the instant invention. In one embodiment, the sealant is the same polymer used to make the polymer support.

In certain embodiments, the nanofibrous polymer support is porous. In one embodiment, the nanofibrous polymer comprises a porosity of about 10% to about 95%. In a further embodiment, the nanofibrous polymer comprises a porosity of about 75% to about 95%.

In other embodiments, the nanofibrous polymer comprises pores with a size distribution ranging from about 2 μm to about 600 μm. In a further embodiment, the nanofibrous polymer comprises pores with a size distribution ranging from about 5 μm to about 475 μm.

In another embodiment, the nanofibrous polymer support comprises polymer nanofibers having a diameter of less than 1 μm. In yet another embodiment, the polymer nanofibers have a diameter of between 50 nm and 1 μm. In certain instances, nanofibrous polymer supports comprise nanofibers having a thickness of less than about less than about 750 nm, or a thickness of between about 50 nm and about 800 nm. In certain other aspects, the nanofibrous polymer scaffold comprises nanofibers having a thickness of between about 100 nm and about 700 nm or between about 200 nm and about 600 nm.

In other embodiments, the polymer nanofibers have a substantially uniform diameter.

In another embodiment, the nanofibrous polymer support comprises a non-woven mat of electrospun nanofibers. In certain embodiments, the nanofibers of the non-woven mat is randomly oriented or specifically oriented.

In other aspects, the nanofibrous polymer supports comprise electrospun nanofibers. Nanofibers prepared by electrospinning provide a nanofibrous polymer support possessing a high surface area to volume ratio and improved mechanical properties relative to hydrogels and other polymeric supports. Although not wishing to be bound by theory, certain nanofibrous polymer supports prepared by electrospinning mimic the fiber diameter and morphological characteristics of collagen in tissues.

In general, electrospinning is a process of producing nanofibers or microfibers of a polymer in which a high voltage electric field is applied to a solution of the polymer. The drawn nanofibers are collected in on a target covering one of the electrodes. By careful regulation of inter-electrode distance, voltage, solvent, and polymer solution viscosity the diameter of the resultant electrospun fibers can be controlled. Optimization of the elecrospinning process results in formation of polymer nanofibers have a substantially uniform diameter.

The term “nanofibrous polymer support” is intended to refer to materials composed of at least one polymeric nanofiber or a plurality of polymeric nanofibers, or combinations thereof. That is, the nanofibrous polymer support is composed of nanofibers composed of a polymer, copolymer, or a blend of polymers or the nanofibrous polymer support comprises two or more compositionally distinct polymeric nanofibers. In certain embodiments, the nanofibrous polymer support is composed of a plurality of uniform thickness nanofibers prepared by an electrospinning process using a solution of one or more polymers. In certain aspects, the polymers are biocompatible, bioabsorbable or biodegradable. In certain embodiments, the nanofibrous polymer support comprises a hydrogel.

In other embodiments, the nanofibrous polymer support of the tissue engineered intervertebral disc is composed of at least one biodegradable and biocompatible polymer support which can be processed by electrospinning to form sub-micron fibers. In certain embodiments, the nanofibrous polymer support is composed of one or more biodegradable biocompatible polyesters. In certain embodiments the biodegradable polyester is a polymer comprising one or more monomers selected from glycolic acid, lactic acid, epsilon-lactone, glycolide, or lactide. The phrase “comprises a monomer” is intended a polymer which is produced by polymerization of the specified monomer, optionally in the presence of additional monomers, which can be incorporated into the polymer main chain. The FDA has approved poly((L)-lactic acid), poly((L)-lactide), poly(epsilon-caprolactone) and blends thereof for use in surgical applications, including medical sutures. An advantage of these tissue engineered absorbable materials is their degradability by simple hydrolysis of the ester linkage in the polymer main chain in aqueous environments, such as body fluids. The degradation products are ultimately metabolized to carbon dioxide and water or can be excreted from the body via the kidney.

In certain embodiments, electrospinning of nanofibers resulted in a scaffold/support composed of uniform, randomly oriented or specifically oriented fibers, as seen by scanning electron microscopy. Following an 8 week incubation in culture medium at 37° C., scaffolds maintained their integrity and three-dimensional structure, while exhibiting no noticeable change in dry weight over the entire culture period.

In certain embodiments, nanofibrous polymer scaffolds/supports are composed of a polymer which is dimensionally stable for at least the time period required to culture the tissue formed using the scaffold.

### Methods of Preparing Tissue Engineered Intravertebral Discs

In one aspect, the invention provides a method of preparing a tissue engineered intervertebral disc comprising the steps of: preparing a nanofibrous biocompatible polymer support comprising a cavity; contacting a suspension of cells with the surface of the support to form a polymer matrix having cells dispersed therein; injecting a hydrogel composition into the cavity; and culturing the cell-polymer matrix in a bioreactor with a culture medium under conditions conducive to growth of cells into a tissue engineered intervertebral disc.

In one embodiment, the invention provides a method of preparing a tissue engineered intervertebral disc further comprising the step of expanding the nanofibrous polymer support thereby increasing interfiber distance.

In another embodiment, the invention provides a method, further comprising the step of compressing the cell-polymer matrix to create cell-cell contact and cell-matrix contact.

In another embodiment, the invention provides a method wherein the nanofibrous polymer support is made by electrospinning.

In certain embodiments, the invention provides a method wherein the nanofibrous polymer support comprises poly(glycolide) (PGA), poly (L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly (ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(3-hydroxybutyrate-co-3-hydroxyvalerate), poly(ethylene-co-vinyl alcohol), and combinations thereof.

In another embodiment, the invention provides a method wherein the hydrogel composition comprises a hydrogel selected from non-biodegradable hydrogels, natural biodegradable hydrogels, and synthetic biodegradable hydrogels. In certain embodiments, the hydrogel composition comprises a hydrogel selected from the following: self-assembly peptide, fibrin, alginate, agarose, hyaluronan, hyaluronic acid, chitosan, chondroitin sulfate, polyethylene oxide (PEO), poly(ethylene glycol) (PEG), collagen type I, collagen type II, and combinations thereof. In other embodiments, the hydrogel composition comprises a hydrogel selected from the following: self-assembly peptide, fibrin, alginate, agarose, hyaluronan, hyaluronic acid, chitosan, chondroitin sulfate, collagen type I, collagen type II, and combinations thereof.

In another embodiment, the invention provides a method wherein the cells are selected from annulus fibrosus cells, nucleus pulposus cells, mesenchymal stem cells, and embryonic stem cells, or combinations thereof. In a further embodiment, each of the annulus fibrosus cells, nucleus pulposus cells, mesenchymal stem cells, and embryonic stem cells dispersed throughout the tissue engineered intervertebral disc is in contact with at least one polymer and at least one other annulus fibrosus cells, nucleus pulposus cell, mesenchymal stem cell, or embryonic stem cell. In another embodiment, each of the annulus fibrosus cells, nucleus pulposus cells, mesenchymal stem cells, and embryonic stem cells dispersed throughout the tissue engineered intervertebral disc is in contact with a plurality of other annulus fibrosus cells, nucleus pulposus cells, mesenchymal stem cells, or embryonic stem cells.

In another embodiment, the invention provides a method wherein the mesenchymal stem cell is isolated from isolated bone marrow, muscle, fat, cord blood, placenta.

In another embodiment, the invention provides a method wherein the cells are stem cells, the culture medium comprises growth factors suitable for annulus fibrosus cell and nucleus pulposus cell differentiation, and the stem cells differentiate to annulus fibrosus cells and nucleus pulposus cells during the culturing step.

In other embodiments, the hydrogel composition is encapsulated by the polymer support. In another embodiment, the cavity is encapsulated by the polymer support. In a further embodiment, the cavity is encapsulated by a sealant. Sealants are selected from nanofibrous polymers of the instant invention. In certain embodiments, the sealant is the same polymer used to make the polymer support.

In other embodiments, the invention provides a method wherein the nanofibrous polymer support is dimensionally stable throughout the culturing step. In certain applications, the nanofibrous polymer scaffold is dimensionally stable for at least about 28 days, at least about 35 days, or at least about 42 days.

In yet another embodiment, the invention provides a method wherein the nanofibrous polymer support is porous. In a further embodiment, the nanofibrous polymer comprises a porosity of about 10% to about 95%. In another further embodiment, the nanofibrous polymer comprises a porosity of about 75% to about 95%.

In other embodiments, the nanofibrous polymer comprises pores with a size distribution ranging from about 2 μm to about 600 μm. In a further embodiment, the nanofibrous polymer comprises pores with a size distribution ranging from about 5 μm to about 475 μm.

In another embodiment, the invention provides a method wherein the nanofibrous polymer support comprises polymer nanofibers having a diameter of less than 1 μm. In a further embodiment, the polymer nanofibers have a diameter of between 50 nm and 1 μm.

In certain embodiments, the invention provides a method wherein the polymer nanofibers have a substantially uniform diameter.

In another embodiment, the invention provides a method wherein the nanofibrous polymer support comprises a non-woven mat of electrospun nanofibers. In a further embodiment, the nanofibers of the non-woven mat is randomly oriented or specifically oriented.

In certain embodiments, the bioreactor suspends the cell-hydrogel-polymer aggregate or tissue engineered intervertebral disc in a moving culture medium. In a further embodiment, the bioreactor comprises a culture chamber in which the cell-polymer matrix and culture medium are placed, and wherein the culture chamber is rotated at a speed sufficient to generate a zero gravity or low gravity mimicking environment in the culture chamber. In another embodiment, the bioreactor provides a dynamic culture medium.

In another aspect, the invention provides a method of forming intervertebral disc in vivo, the method comprising the steps of: preparing the tissue engineered intervertebral disc of the invention; and inserting the tissue engineered intervertebral disc into a subject at the position suitable for formation of new intervertebral disc. In one embodiment, the subject is a mammal. In a further embodiment, the subject is a human.

In one embodiment, the invention provides a method wherein the tissue engineered intervertebral disc is inserted into a region of existing damaged intervertebral disc in the subject.

In another aspect, the invention provides a method of treating intervertebral disc damage, the method comprising the steps of: preparing the tissue engineered intervertebral disc of the invention; and inserting the tissue engineered intervertebral disc into a subject at the location of the damaged intervertebral disc.

In certain embodiments, the subject suffers from osteoarthritis arthritis, rheumatoid arthritis, developmental disorders, or traumatic injury each of which induced intervertebral disc damage.

In another embodiment, the location of damaged intervertebral disc is a spine. In a further embodiment, the location of damaged intervertebral disc is an inter-vertebrae.

In other embodiments, the intervertebral disc damage is abrasion, tear, wear, or compression.

In another aspect, the invention provides a method for treating intervertebral disc damage, the method comprising the steps of: harvesting annulus fibrosus cells, nucleus pulposus cells, mesenchymal stem cells, or embryonic stem cells from a subject; preparing tissue engineered intervertebral disc of the invention, wherein the cells are the annulus fibrosus cells, nucleus pulposus cells, mesenchymal stem cells, or embryonic stem cells harvested from the subject; implanting the tissue engineered intervertebral disc in the subject in a locus having damaged intervertebral disc.

In still another aspect, the invention provides a method for cosmetic or reconstructive surgery, the method comprising the steps of: preparing the tissue engineered intervertebral disc of the invention; and inserting the tissue engineered intervertebral disc into a subject.

In yet another aspect, the invention provides a method for cosmetic or reconstructive surgery, the method comprising the steps of: harvesting annulus fibrosus cells, nucleus pulposus cells, mesenchymal stem cells, or embryonic stem cells from a subject; preparing tissue engineered intervertebral disc of the invention, wherein the cells are the annulus fibrosus cells, nucleus pulposus cells, mesenchymal stem cells, or embryonic stem cells harvested from the subject; and implanting the tissue engineered intervertebral disc in the subject in a locus having damaged intervertebral disc.

In one embodiment, the spine is being reconstructed or cosmetically reconfigured, and the tissue engineered intervertebral disc is implanted in the spine.

In another aspect, the invention provides a method of preparing tissue engineered intervertebral disc comprising the steps of: preparing a nanofibrous biocompatible polymer support comprising a cavity; contacting a suspension of cells with the surface of the support to form a polymer matrix having cells dispersed therein; injecting a hydrogel composition into the cavity; and culturing the cell-polymer matrix in a bioreactor with a culture medium under conditions conducive to cell growth and differentiation to tissue engineered tissue.

In yet another aspect, the invention provides a method of preparing a tissue engineered tissue comprising the steps of: preparing a nanofibrous biocompatible polymer support comprising a cavity; expanding the nanofibrous polymer support thereby increasing interfiber distance; contacting a suspension of cells with the support to form a polymer matrix having cells dispersed therein; injecting a hydrogel composition into the cavity; culturing the compressed cell-polymer matrix in a bioreactor with a culture medium under conditions to conducive cell growth and differentiation to tissue engineered tissue.

In one embodiment, the present invention provides methods of treating disease and/or disorders or symptoms thereof which comprise administering a nanofibrous polymer-hydrogel-cell amalgam, to a subject (e.g., a mammal such as a human). More particularly, the present invention provides methods of treating damaged or destroyed disc (knee, ankle, hand, wrist, elbow, shoulder, hip, or intervertebrae) wherein the damage is abrasion, tear, wear, or compression, by inserting tissue engineered intevertebral discs herein at the locus of disc damage or destruction in the subject. Thus, for example, a subject suffering from arthritis of the spine may have damaged or destroyed some or all of the discs. The methods of the invention provide for treatment by inserting tissue engineered intevertebral discs at the point of damage to replace or repair the damaged disc.

In certain other aspects, engineered intevertebral disc provided herein is administered to a subject (e.g., a mammal such as a human) to provide desirable reconstructive or cosmetic benefit to the subject. Thus, for example, a subject sustained an injury which caused damage or destruction of the spine. The methods of the invention provide for reconstruction or cosmetic enhancement of the spine by inserting a formed engineered intevertebral disc into the damaged spine thereby improving the function or aesthetics of the spine.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used “cosmetic surgery” or “reconstructive surgery” is intended herein to refer to surgical procedures intended to modify or improve the appearance of a physical feature, irregularity, or defect.

Contacting the Cells with the Polymer/Hydrogel Amalgam

In certain methods, a nanofibrous polymer non-woven mat is electrospun onto a rotary rod to from a hollow nanofibrous tube with a desired thickness and then cut into a desired shape, including a cavity. Polymer sealants cover the two ends of the cavity after fluffy nanofibers is stuffed in the cavity. The terms “fluffy nanofiber” and “cottonball nanofiber” are used interchangeably. A hydrogel composition mixed with cells is added to the cavity to form the nanofibrous polymer hydrogel amalgam. In certain embodiments, a solution of cells is then applied to the surface of the amalgam using a spinner-flask to form a cell-polymer-hydrogel matrix. During culturing the cells diffuse through the thickness of the polymer/hydrogel amalgam to form a cell-polymer-hydrogel matrix. In certain embodiments, the cells are selected from annulus fibrosus and nucleus pulposus cells, mesenchymal stem cells, or embryonic stem cells.

In certain instances, a cell culture tube is charged with the nanofibrous polymer substrate and then a solution of cells is added to the cell culture tube. The cell-polymer-hydrogel aggregate is then cultured statically or dynamically in the tube to generate the intervertebral disc. As used herein, “statically cultured,” “cultured in a static environment,” or like terms are intended to refer to culturing conditions in which the culture medium is not moving relative to the cell-polymer-hydrogel matrix. As used herein, “dynamically cultured,” “cultured in a dynamic environment,” or like terms are intended to refer to culturing conditions in which the culture medium is moving relative to the cell-polymer-hydrogel matrix. In certain embodiments, the culture medium is a chondrogenic medium preferably comprising one or more growth factors. The dynamic or static culturing is conducted at 37° C. in a humidified 5% carbon dioxide atmosphere. In certain methods comprising static culturing, the culture vessel is a cell culture tube, a culture medium and the cell-substrate aggregate are charged in the cell culture tube, and the mixture maintained at 37° C. under a humidified 5% carbon dioxide atmosphere. Culturing using a culture tube is referred to herein as “static” culturing.

In other methods, a nanofibrous polymer non-woven mat is expanded to introduce more porosity in the nanofibrous polymer scaffold. That is, in certain embodiments, an electrospun polymer mat is plucked, combed, teased or otherwise mechanically treated to increase the inter-fiber distances in the mat such that the expanded nanofibrous polymer scaffold has a “cotton ball” or fluffy appearance. In certain embodiments, the “cotton ball” polymer or mixture of polymers is added into the inner layer of the nanofibrous polymer support of the intevertebral disc. The “cotton ball” nanofibers with a loosened fiber structure serve the roles of mechanical reinforcement and biological enhancement. In another embodiment, the “cotton ball” polymer or mixture of polymers, in the inner layer of the intervertebral disc, forms an amalgam with a hydrogel. The expanded mat is then contacted with a solution of cells. Although not wishing to be bound by theory, the increased inter-fiber distances present in the expanded nanofibrous polymer scaffold permits creates more apertures through which the cells can disperse into the expanded nanofibrous polymer-gel amalgam thereby providing a more uniform distribution of cells throughout the amalgam.

In certain embodiments, the polymer-hydrogel-cell matrix is cultured for between 1 and about 10 days in a static or dynamic environment to generate increased integration of the polymer-hydrogel-cell matrix. In certain other embodiments the polymer-hydrogel-cell matrix is cultured in a static or dynamic vessel for between 2 to 10 days or between 3 and 7 days. Although not wishing to be bound by theory, the static or dynamic culturing period is believed to allow the cells to generate an extracellular matrix which holds the fibers of the nanofibrous polymer support in position.

In certain aspects, after dynamic or static culturing, the polymer-hydrogel-cell matrix is transferred to a bioreactor for additional culturing of up to about 42 days during which time the intevertebral disc is formed. The term “bioreactor” is intended to refer to vessels suitable for culturing cells or polymer-hydrogel-cell matrixes, wherein the bioreactor improves delivery of nutrients and removal of waste products associated with cellular maintenance and development. Preferred bioreactor devices and vessels in which one or more biological or biochemical processes can be conducted under closely monitored and controlled conditions, e.g., environmental and/or operating conditions can be regulated by an operator. Certain bioreactors are devices in which the temperature, acidity (pH), pressure, nutrient supply, atmosphere, and/or removal of waste can be regulated by an operator or a control device. Bioreactors suitable for use in the methods of making tissue engineered IVD provide a dynamic growth environment. The terms “dynamic,” “cultured in a dynamic environment” and the like are intended to refer to culturing conditions in which the culture medium experiences at least one translational, rotational, or other mechanical force capable of causing the culture medium to flow or otherwise be translated in the bioreactor culture chamber. In general, bioreactors which generate movement of the culture medium relative to the polymer-hydrogel-cell matrix or the tissue engineered IVD present in the bioreactor chamber are preferred. In certain aspects, the bioreactor is selected from devices which direct a continuous flow of a culture medium or other fluid at the cell-polymer-hydrogel aggregate or tissue charged into the bioreactor culture chamber. In certain embodiments, the bioreactor is selected from spinner-flask bioreactors, rotating-wall vessel bioreactors, hollow fiber bioreactors, direct perfusion bioreactors, bioreactors that apply a controlled direct mechanical force to the cell-polymer aggregate or tissue, and other bioreactor designs that deliver continuous fluid flow to the cell-polymer aggregate or tissue. In certain other aspects, the bioreactor is a rotating bioreactor having a chamber charged with the cell-substrate aggregate and culture medium. In another embodiment, the chamber is shaped so as to form a cell-polymer-hydrogel that is conical. The bioreactor is rotated about the central axis at a rate sufficient to offset the force of gravity. Culturing using a rotating bioreactor such as a rotating bioreactor is referred to herein as “dynamic” culturing.

In certain aspects the culture medium is formulated to support the target engineered tissue. Thus, where IVD is the target tissue, the culture medium is a chemically defined medium appropriate for maintenance of annulus fibrosus and nucleus pulposus cells or inducing differentiation of mesenchymal stem cells to annulus fibrosus and nucleus pulposus cells. Certain chemically defined media comprise one or more growth factors which regulate and/or promote annulus fibrosus and nucleus pulposus cell formation, development or growth.

In certain methods provided herein, the culture medium comprises one or more growth factors suitable for promoting growth and development of annulus fibrosus and nucleus pulposus cells and the differentiation of stem cells into annulus fibrosus and nucleus pulposus cells. In certain aspects, the growth factors are selected from transforming growth factors (TGF), insulin-like growth factors (IGF), bone morphogenic proteins (BMP), fibroblast growth factors (FGF), and combinations thereof. In certain methods, the growth factors are selected from IGF-1, TGF-131, TGF-133, BMP-7 and combinations thereof.

The invention will be further described in the following examples. It should be understood that these examples are for illustrative purposes only and are not to be construed as limiting this invention in any manner.

### Example 1

### Isolation and Culture of Bone Marrow-Derived hMSCs

With approval from the Institutional Review Board of Thomas Jefferson University, bone marrow-derived hMSCs were obtained from the femoral heads of subjects undergoing total hip arthroplasty, and processed as previously described (Noth U, et al. J Orthop Res 2002; 20:1060-9; Haynesworth S E, et al. Bone 1992; 13:81-8; and Wang M L, et al. J Orthop Res 2002; 20:1175-84). Briefly, whole bone marrow was curetted from the exposed cutting plane of the femoral neck, washed extensively in Dulbecco's Modified Eagle's medium (DMEM; BioWhittaker, Walkersville, Md.), separated from contaminating trabecular bone fragments and other tissues using a 20-gauge needle attached to a 10-cc syringe, and cultured in DMEM, 10% fetal bovine serum (FBS) from selected lots (Caterson E J, et al. Mol Biotechnol 2002; 20:245-56), and antibiotics (50 μg/mL streptomycin, 50 IU/mL of penicillin) at a density of 4×10⁵ cells/cm². Forty-eight hours post-plating, tissue culture flasks were washed twice with phosphate-buffered saline (PBS) to remove non-adherent cells. Medium changes were made every 3-4 days. Subconfluent cell monolayers were dissociated using 0.25% trypsin and either passaged or utilized directly for study.

### Example 2

### Fabrication of Electrospun Nanofibrous PLLA Scaffolds

Nanofibrous scaffolds were fabricated according to an electrospinning process described previously (Li W J, et al. J Biomed Mater Res 2003; 67A:1105-14). Briefly, PLLA polymer was dissolved in an organic solvent mixture (10:1) of chloroform and N,N, dimethylformamide (DMF) at a final concentration of 0.14.5 g/mL. The polymer solution was delivered through the electrospinning apparatus at a constant flow rate of 0.4 mL/h under an applied 0.8 kV/cm charge density, resulting in a 144 cm² mat with an approximate thickness of 1 mm. To remove residual organic solvent, the non-woven polymer mat was placed within a vacuum chamber for 48 h, and subsequently stored in a dessicator. Prior to cell seeding, nanofibrous scaffolds were fashioned from the electrospun mat, sterilized by ultraviolet irradiation for 30 min per side in a laminar flow hood, and pre-wetted for 24 h in Hanks' Balanced Salt Solution.

### Example 3

### Fabrication of Intervertebral Disc (IVD) Constructs

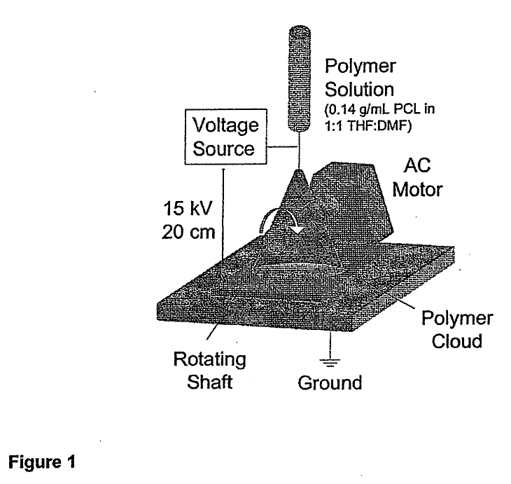

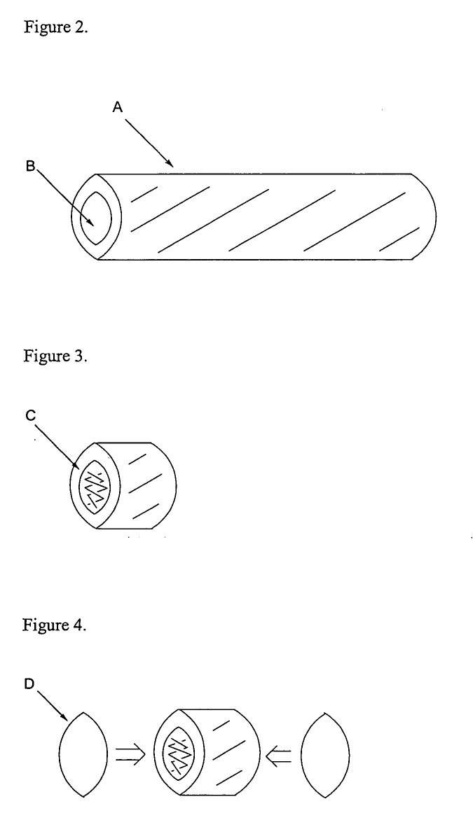

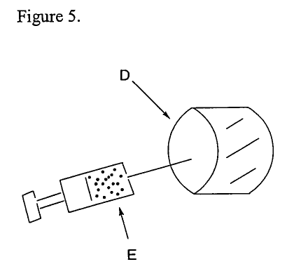

To make IVD constructs, PLLA nanofibers were electrospun onto a rotating rod (shaft) to produce homogeneous, non-woven or specifically oriented nanofibrous mats (FIG. 1), whose shape was dependent on the mechanical requirements for a construct. After pulling out the rod, a long hollow nanofibrous tube (FIG. 2) with the outer diameter of 1.1 cm and the inner diameter of 1.0 cm was produced. Nanofibrous rings with the height of 0.5 cm were obtained from cutting the nanofibrous tube into sections (FIG. 3). The open-to-outside ring was sealed with a circular nanofibrous mat with the diameter of 1.1 cm on each end of the ring after being inserted with fluffy nanofibers (FIG. 4). The inserted nanofibers with a loosened fiber structure serve the roles of mechanical reinforcement and biological enhancement.

A hydrogel such as hyaluron gel was mixed with nucleus pulposus cells isolated from human IVD, and was injected into the empty space with pre-occupied fluffy nanofibers, encapsulated with nanofibrous mats. The hydrogel injection continued until the entire space was filled with hydrogel, creating a stiff, compression-resisted IVD construct due to the mechanical tension generated in the encapsulated space (FIG. 5).

### Example 4

### Culture of IVD Constructs

Nanofiber-hydrogel composite based IVD pre-seeded with nucleus pulposus cells were placed in the spinner-flask bioreactor and cultured in a continuously stirred cell culture medium containing human annulus fibrosus cells. IVD constructs were transferred to cell culture plates or rotary wall vessel bioreactors for continuous growth and tissue maturation after annulus fibrosus cells were evenly attached onto the surface of the IVD constructs in the spinner-flaks bioreactor. Mesenchymal stem cells were also examined as a replacement for nucleus pulposus and annulus fibrosus cells.

### Example 5

### Biological Evaluation of Tissue Engineered IVD

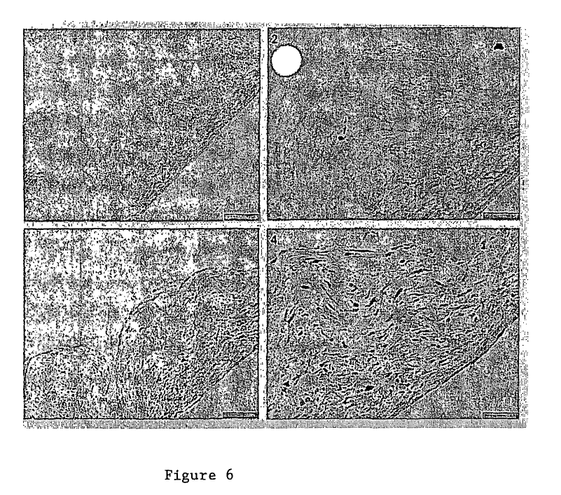

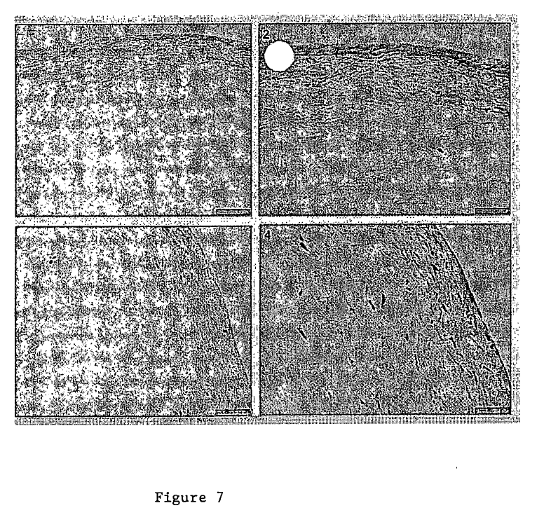

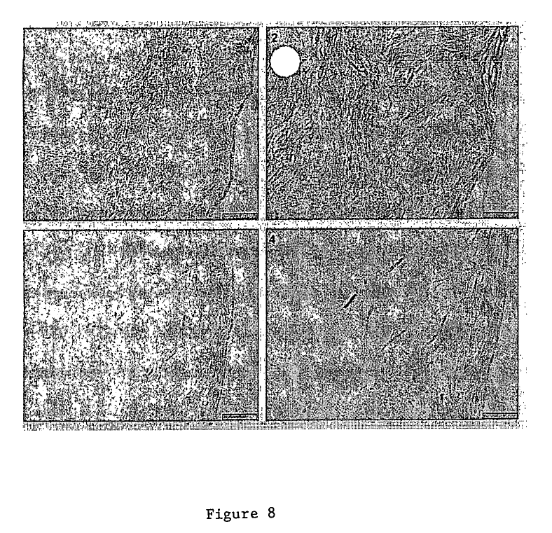

Histological staining was performed at 7 (FIG. 6), 14 (FIG. 7) and 28 days (FIG. 8). H&E staining demonstrated uniform cell loading in the AF at the early time points. With increasing periods in culture the cells began to elongate and layer in a concentric fashion, similar to the microarchitecture of a native AF. The native AF is organized in a series of centric fibrous-like rings that impart much of the tensile strength to the disc. Increases in ECM deposition are also seen on the sections with complete filling of the nanofiber pores within the AF by Day 28. Initially cells of the NP appeared to be sparse with little ECM deposition. The small number of cells at the early time points may be a result of sectioning artifact as insufficient ECM had been produced at this early time to support individual cells during the sectioning process. Later in the culture period, after deposition of a more mature ECM, cells appeared rounded and encapsulated in the ECM—a notable difference from the layered cells in the region of the AF.

Alcian blue staining allows for visualization of a proteoglycan rich ECM. The intensity of the staining in the IVD construct increased throughout the 28 day culture period with the most intense staining observed in a ring like fashion of the AF region. Alcian blue staining of the NP appeared amorphous without distinct organization. This staining pattern correlates with the intended structural design of the construct, which is an organized ring-like barrier containing a relatively amorphous center. Of interest here is the integrated transition between the outer AF and inner NP. The relatively seamless transition between the two regions in our construct closely mimics that seen in native human disc where there is no distinct division between the two disc regions.

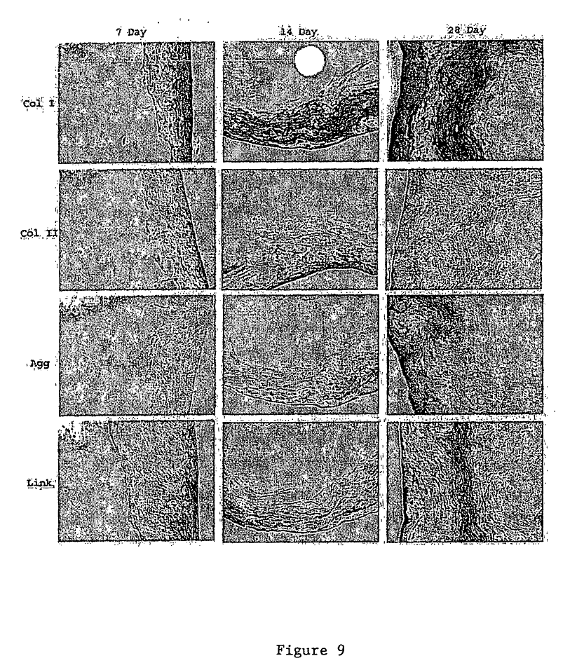

Immunohistochemical staining for known IVD ECM components was performed (FIG. 9). Sections were positive for col II, col IX, aggrecan and link protein. The staining pattern was similar to that seen in the alcian blue sections with increasing intensity over the 28 day culture period. Notable deposition of col II, IX, aggrecan and link protein were noted in the immediate pericellular area with increasing deposition in the surrounding construct over 28 days. Positive staining of these antibodies confirms the deposition of a ECM similar to that of a native IVD. Col II and IX demonstrate the presence of a fibrillar collagen network supplemented by a proteoglycan matrix as visualized with intense aggrecan and link protein staining. Negative controls performed without primary antibody confirmed specificity of the antibodies.

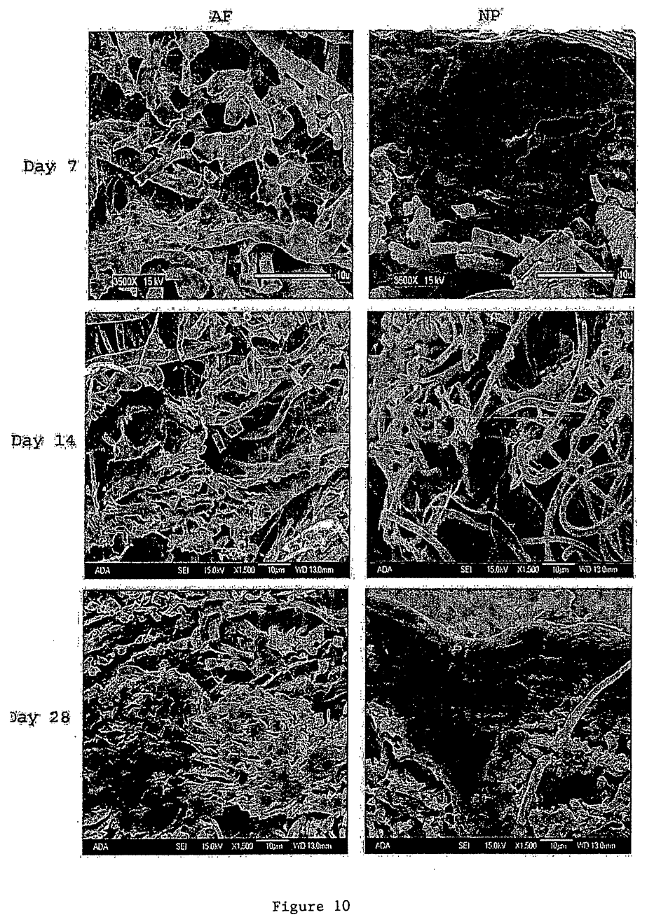

Scanning electron microscopy demonstrated uniform cell distribution and cell adhesion in the nanofibrous scaffold similar to that of previously reported findings in cartilage TE studies (Li, W. J., et al., Biomaterials 2005:26:599-609). At the earliest time point, rounded cells were adherent to individual nanofibers and demonstrated minimal ECM deposition in both the AF and NP. Over time, abundant ECM is formed and fills the small pores between nanofibers and larger void within the NP (FIG. 10). Nanofiber architecture remained in tact for the duration of the experiment and ultimately became intimately associated with the surrounding extracellular matrix.

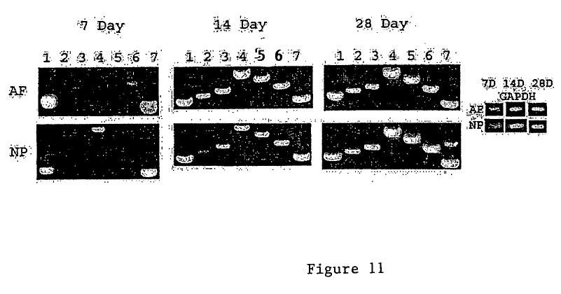

RT-PCR was performed to assess the presence of key messages necessary for ECM production in the IVD (FIG. 11). Specifically, col I, col II, col IX, col X, col XI, aggrecan and COMP were all probed and found to be present in full compliment by Day 14 with col I and COMP expression occurring as early as 7 days. Of particular interest here is the ability to express and maintain expression of col II and col IX. The difficulty of expressing col II and col IX in culture has been well established and requires cell culture in a three dimensional microenvironment. In the present culture system expression of high levels of col II was obtained and maintained the high level of expression over the entire experimental course.

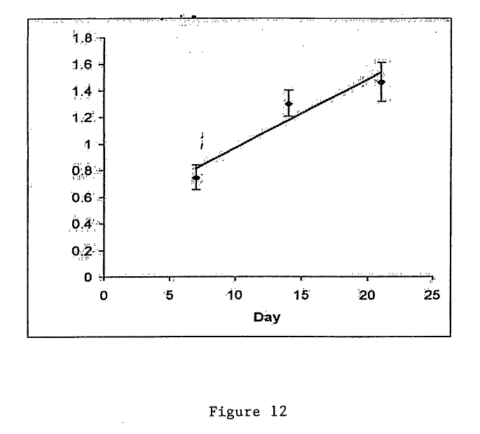

Failure of the IVD is often documented clinically with decreased signal intensity on T2 MRI images, signifying decreased hydration state of the disc. Proteoglycan expression is critical for maintaining a hydrated state of the disk so the proteoglycan expression was quantified in the TE construct using the blyscan method. Proteoglycan expression was evident as early as 7 days of culture and significantly increased over the 28 day culture period (FIG. 12).

The cellular morphological characteristics in the two regions of the disc suggest a divergence in behavioral properties based on physical microenvironment. This variation could result from separate mechanical forces exposed to the cells in each region, different diffusion properties for nutrient and O₂ supply or cell loading density.

The MSC's presently used were able to adhere to the nanofibrous polymer-hydrogel amalgam, proliferate and differentiate and secrete a proteoglycan rich ECM with a protein expression profile similar to that of a native IVD. The use of MSC's as a cell source for IVD reconstruction has been previously reported and it is likely that they will be invaluable in developing a tissue engineered-IVD. The ability of these cells to produce such a proteoglycan-rich matrix in the present construct is of great importance as it addresses the common theme in disc degeneration, specifically loss of proteoglycan production and dehydration of the disc.

### Example 6

### Culture Cell-Polymer-Hydrogel Aggregate in a Rotating Vessel Wall Bioreactor

The cell-nanofiber-hydrogel composite is placed in a rotating vessel wall bioreactor for next 42 days. The rotation speed of a rotating-wall vessel bioreactor is controlled to maintain the cell-nanofiber-hydrogel composite stay in the situation of floating in the medium. The cell-nanofiber-hydrogel composite is cultured in the culture medium and half the volume of the cell culture medium is replaced every three days.

### Example 7

### Physical and Biochemical Analysis Methods

7.1. Scanning Electron Microscopy (SEM)

For each condition, two cell-polymer-hydrogel constructs are fixed in 2.5% glutaraldehyde, dehydrate through a graded series of ethanol, vacuum dry, mount onto aluminum stubs, and sputter coat with gold. Samples are examined using a scanning electron microscope (S-4500; Hitachi, Japan) at an accelerating voltage of 20 kV.

7.2. Reverse Transcription Polymerase Chain Reaction (RT-PCR) Analysis

Total cellular RNA are extracted using Trizol Reagent according to the manufacturer's protocol. Concentrations of RNA samples are estimated on the basis of OD₂₆₀. RNA samples are reverse transcribed using random hexamers and the SuperScript First Strand Synthesis System. PCR amplification of cDNA is carried out using AmpliTaq DNA Polymerase and the gene-specific primer sets. The housekeeping gene, glyceraldehyde 3-phosphate dehydrogenase (GAPDH), is used as a control for RNA loading of samples. PCR products are analyzed electrophoretically.

7.3. Cryoembedding and Sectioning

For each condition, two constructs are harvested, fix in 4% PBS-buffered paraformaldehyde for 15 min, wash three times with PBS, infiltrate with 20% sucrose, embed with Tissue-Tek O.C.T Compound, and cryosection at 8 mm thickness using the Leica CM 1850 (Bannockburn, Ill.) cryostat microtome.

7.4. Histological Analysis

Cell-polymer-hydrogel constructs are harvested, rinsed, fixed, dehydrated, and embedded. A 8 μm-thick section is prepared and stained with H&E and Alcian blue for cell morphology and proteoglycan, respectively.

7.5. Immunohistochemical Analysis

Immunohistochemistry is used to detect aggrecan, collagen type II, and link protein, in cell-polymer-hydrogel constructs. Sections are pre-digested in chondroitinase A/B/C before they are incubated in primary antibody. Antigen-antibody complexes are detected colorimetrically using the Broad Spectrum Histostain-SP Kit; sections are counterstained with hematoxylin.

### INCORPORATION BY REFERENCE

All patents, published patent applications, and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A tissue engineered intervertebral disc, comprising
a nanofibrous polymer support comprising one or more polymer nanofibers;
a hydrogel composition comprising at least one or more hydrogel materials; and
a plurality of cells which are dispersed throughout the tissue engineered intervertebral disc.

2. A tissue engineered intervertebral disc comprising at least one inner layer and an exterior layer, wherein:
the exterior layer comprises a nanofibrous polymer support comprising one or more polymer nanofibers;
the at least one inner layer comprises a hydrogel composition comprising at least one or more hydrogel materials; and
a plurality of cells which are dispersed throughout the tissue engineered intervertebral disc.

3. A tissue engineered intervertebral disc comprising at least one inner layer and an exterior layer, wherein:
the exterior layer comprises a nanofibrous polymer support comprising one or more polymer nanofibers;
the at least one inner layer comprises a hydrogel composition comprising at least one or more hydrogel materials and one or more polymer nanofibers; and
a plurality of cells which are dispersed throughout the tissue engineered intervertebral disc.

4. The tissue engineered intervertebral disc of claim 1, wherein the nanofibrous polymer support is made by electrospinning.

5. The tissue engineered intervertebral disc of claim 1, wherein the nanofibrous polymer support comprises poly(glycolide) (PGA), poly (L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly (ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(3-hydroxybutyrate-co-3-hydroxyvalerate), poly(ethylene-co-vinyl alcohol), and combinations thereof.

6. The tissue engineered intervertebral disc of claim 1, wherein the nanofibrous polymer support comprises biodegradable poly(α-hydroxy ester) polymers.

7. The tissue engineered intervertebral disc of claim 6, wherein the nanofibrous polymer support comprises polymers selected from poly(lactic acid) (PLA), poly(glycolide) (PGA), and poly(lactide-co-glycolide) (PLGA), and combinations thereof.

8. The tissue intervertebral disc of claim 1, wherein the nanofibrous polymer support comprises poly(glycolide) (PGA), poly(lactide-co-glycolide) (PLGA), poly(L-lactide) (PLLA), poly(D,L-lactide) (P(DLLA)), poly(ε-caprolactone) (PCL), and combinations thereof.

9. The tissue engineered intervertebral disc of claim 1, wherein the hydrogel composition comprises a hydrogel selected from non-biodegradable hydrogels, natural biodegradable hydrogels, and synthetic biodegradable hydrogels.

10-28. (canceled)

29. A method of preparing a tissue engineered intervertebral disc comprising:
preparing a nanofibrous biocompatible polymer support comprising a cavity;
contacting a suspension of cells with the surface of the support to form a polymer matrix having cells dispersed therein;
injecting a hydrogel composition into the cavity; and
culturing the cell-polymer matrix in a bioreactor with a culture medium under conditions conducive to growth of cells into a tissue engineered intervertebral disc.

30. The method of claim 29, further comprising the step of expanding the nanofibrous polymer support thereby increasing interfiber distance.

31. The method of claim 29, further comprising the step of compressing the cell-polymer matrix to create cell-cell contact and cell-matrix contact.

32-59. (canceled)

60. A method of forming intervertebral disc in vivo, the method comprising:
preparing the tissue engineered intervertebral disc of claim 1; and
inserting the tissue engineered intervertebral disc into a subject at the position suitable for formation of new intervertebral disc.

61. The method of claim 60, wherein the tissue engineered intervertebral disc is inserted into a region of existing damaged intervertebral disc in the subject.

62. A method of treating intervertebral disc damage, the method comprising:
preparing the tissue engineered intervertebral disc of claim 1; and
inserting the tissue engineered intervertebral disc into a subject at the location of the damaged intervertebral disc.

63-66. (canceled)

67. A method for treating intervertebral disc damage, the method comprising:
harvesting annulus fibrosus cells, nucleus pulposus cells, mesenchymal stem cells, or embryonic stem cells from a subject;
preparing tissue engineered intervertebral disc by the method of claim 29, wherein the cells are the annulus fibrosus cells, nucleus pulposus cells, mesenchymal stem cells, or embryonic stem cells harvested from the subject;
implanting the tissue engineered intervertebral disc in the subject in a locus having damaged intervertebral disc.

68. A method for cosmetic or reconstructive surgery, the method comprising:
preparing the tissue engineered intervertebral disc of claim 1; and
inserting the tissue engineered intervertebral disc into a subject.

69. A method for cosmetic or reconstructive surgery, the method comprising the steps of
harvesting annulus fibrosus cells, nucleus pulposus cells, mesenchymal stem cells, or embryonic stem cells from a subject;
preparing tissue engineered intervertebral disc by the method of claim 29, wherein the cells are the annulus fibrosus cells, nucleus pulposus cells, mesenchymal stem cells, or embryonic stem cells harvested from the subject;
implanting the tissue engineered intervertebral disc in the subject in a locus having damaged intervertebral disc.

70. (canceled)

71. A method of preparing tissue engineered intervertebral disc comprising the steps of:
preparing a nanofibrous biocompatible polymer support comprising a cavity;
contacting a suspension of cells with the surface of the support to form a polymer matrix having cells dispersed therein;
injecting a hydrogel composition into the cavity; and
culturing the cell-polymer matrix in a bioreactor with a culture medium under conditions conducive to cell growth and differentiation to tissue engineered tissue.

72. A method of preparing a tissue engineered tissue comprising the steps of:
preparing a nanofibrous biocompatible polymer support comprising a cavity;
expanding the nanofibrous polymer support thereby increasing interfiber distance;
contacting a suspension of cells with the support to form a polymer matrix having cells dispersed therein;
injecting a hydrogel composition into the cavity; and
culturing the compressed cell-polymer matrix in a bioreactor with a culture medium under conditions to conducive cell growth and differentiation to tissue engineered tissue.

73. A method of forming intervertebral disc in vivo, the method comprising:
preparing a tissue engineered intervertebral disc by the method of claim 29; and
inserting the tissue engineered intervertebral disc into a subject at the position suitable for formation of new intervertebral disc.

74. A method of treating intervertebral disc damage, the method comprising:
preparing a tissue engineered intervertebral disc prepared by the method of claim 29; and
inserting the tissue engineered intervertebral disc into a subject at the location of the damaged intervertebral disc.
